(19) United States
(12) Patent Application Publication
(10) Pub. No.: US 2011/0137211 A1
(43) Pub. Date: Jun. 9, 2011

(54) OPTICAL DEVICE AND TRAINING METHOD FOR CORRECTING POSTURE

(76) Inventors: Allan Weisberg, Ardsley, NY (US); Jason Sapan, White Plains, NY (US)

(21) Appl. No.: 12/959,065

(22) Filed: Dec. 2, 2010

Related U.S. Application Data
(60) Provisional application No. 61/265,791, filed on Dec. 2, 2009.

(51) Int. Cl. A61B 5/103

(52) U.S. Cl. 600/587

(57) ABSTRACT

A device and a method whereby a computer user seated at a first predetermined position can be provided with a continuous visual indication of the user's spinal posture with respect to the computer monitor. A device is provided which shows either a first indication or a second indication, as a function of the angle at which it is viewed by the user. The first indication is indicative of a viewing angle with a most preferred spinal posture, and the second indication is indicative of a viewing angle with a less preferred spinal posture. The device is placed in a second predetermined position on the computer monitor, so that the user will see either the first or the second indication, depending on whether the user is currently seated with the most preferred or the less preferred spinal posture. Alternatively, an indicator or an appearance exhibited by the device may depend on the angle at which it is viewed by the viewer, rather than the user's spinal posture per se.

### CROSS REFERENCE TO RELATED APPLICATION

The present application is based upon and claims priority of provisional application No. 61/265,791, filed Dec. 2, 2009, incorporated by reference.

### FIELD OF THE INVENTION

The present invention relates to an optical device that provides to computer user with feedback regarding the computer user's posture, and to a method of improving the user's posture, especially while seated at a computer or in another similar situation.

### BACKGROUND OF THE INVENTION

A person sitting in front of a monitor, such as a computer monitor, a video monitor, or other similar device (hereinafter, for convenience, “computer user” or simply “user” or “person”) for prolonged or repetitive periods of time, e.g., several hours over several days, often will be unaware of their gradual, accumulative transition from an upright proper posture that is in alignment with gravity center, to a somewhat slouched, incorrect and forward head posture. Over a period of time this can result in the development of a poor postural habit which can cause ongoing chronic situations. As a result, the person may suffer from neuromuscular and spinal related problems including neck and back pain and/or, possibly, the development of spinal disc degeneration, without ever realizing the underlying cause of the problem. In addition, incorrect posture and poor computer workstation ergonomics continue to contribute to both the increased incidence and costs related to Repetitive Stress Injuries (RSI) in the computer workplace. In addition, poor posture degrades a person's appearance and confidence.

Therefore, there is a need and value for a device for informing a person that his/her posture, for example while using a computer or watching a video monitor, is not ergonomically correct, and a method of training to improve the person's posture.

### SUMMARY OF THE INVENTION

In view of the foregoing, and in accordance with an aspect of the present invention, there is provided an optical device that provides to a person visual feedback regarding his/her spinal posture alignment using, for example, a lenticular lens or other similar functional device. The optical device may be placed at a predetermined position upon a computer monitor, video monitor or other similar device (hereinafter, for convenience “monitor”) for example in the central top area of the monitor. In addition, the optical device is preferably adhered or mounted to the monitor in a manner that allows for relatively easy angular adjustments of the optical device.

As an aspect of the present invention, the optical device and/or its specific placement instructions for its correct placement onto the monitor, allows for adjustable compensations to be made for the different heights of its user, varying changes in available light sources, as well as for different size or shaped monitors.

As another aspect of the present invention, the visual feedback of the optical device may be disposed within a series of interlaced images or as a series of separate images as in a lenticular lens or hologram.

As a feature of the present invention, the person may view images, but only one at a time, displayed by the lenticular lens depending on the person's viewing angle relative to the optical device.

As another feature, the optical device can either be permanently mounted, or removed from the monitor and later reattached to the same or different monitor in order to enable one to employ the device at different times and/or with different computer monitor systems.

As a further aspect of the present invention, the optical device may display two or more images in order to provide sufficient visual feedback to a user regarding the user's posture, i.e., correct posture vs incorrect posture.

These and other aspects, features and benefits of the present invention are discussed in the detailed description below, which discloses one embodiment of the invention.

### BRIEF DESCRIPTION OF THE DRAWINGS

The following detailed description, given by way of example and not intended to limit the present invention solely thereto, will best be appreciated in conjunction with the accompanying drawings in which:

FIG. 1 is an exemplary image that may be displayed by the optical device when viewed by a person whose posture is within a proper ergonomic and correct spinal alignment relationship with their monitor;

FIG. 2 is an exemplary image that may be displayed by the optical device when viewed by a person whose posture is partially out of the proper ergonomic and correct spinal alignment relationship with their monitor.

FIG. 3 is an exemplary image that may be displayed by the optical device when viewed by a person whose posture and correct ergonomic and/or correct spinal alignment relationship with their monitor is dramatically, or grossly incorrect;

FIG. 4 is a schematic illustration that depicts the operation of a lenticular lens when viewed by a person disposed at different angular locations relative to the optical device;

FIG. 5 is a schematic illustration of a person using the optical device; and

FIG. 6 is a schematic illustration that shows an example of proper placement of the optical device on a monitor.

### DETAILED DESCRIPTION OF AN EMBODIMENT

Disclosed is an optical device that employs lenticular lens technology. As is well known, a lenticular lens is composed of a series of parallel cylindrical lenses formed in a single sheet of clear plastic or other suitable material in a corduroy pattern. Multiple images within the lenticular lens are formed from multiple whole original images divided into a series of horizontal strips like a Venetian blind. The original images are interlaced into one resulting image combining all of the original images into a matrix. If three original images are employed, then the final matrix of the resulting image consists of the three original images interlaced together in an alternating manner, where the three separate images “A”, “B” and “C” are assembled in the order A1B1C1, A2B2C2, A3B3C3, etc., until the entirety of all three images are completed as a series of horizontal strips. Alternatively, the assembled matrix of horizontal images can also be printed directly onto the rear side of a lenticular lens. Other known suitable manners of producing a lenticular lens also may be employed.

The image presented to the user by the optical device changes as a function of the user's viewing angle so that the user sees only one image at a time depending on his/her viewing angle relative to the location of the optical device. As described in further detail below, the particular image seen by the user in the optical device changes to reflect changes in the viewer's posture. As a result, the perceived visual image changes in relation to changes in the viewer's postural movement during his/her use of the monitor.

In accordance with one embodiment, the optical device may be designed to display to a user different colors indication the quality of depending on the user's posture and spinal alignment with proper placement of the optical device. Preferably, the colors are specifically selected to reinforce feedback provided to the user. In one version, the colors provided may be those of a traffic light, that is, green for proper spinal posture alignment, yellow for partially improper spinal posture alignment, and red for dramatically poor spinal posture alignment.

In addition to or instead of providing different colors, the optical device may display different respective images which change accordingly to exemplify good, bad, and terrible spinal postures, depending on the respective positional changes of the user's posture. Hence, different images in different colors may be viewed by the user depending on whether the user's spinal posture alignment is ergonomically correct, ergonomically partially incorrect or ergonomically completely incorrect.

In accordance with the disclosed system a user who is sitting in a healthy and ergonomically correct manner sees within the optical device a first green image thereby providing to the user feedback that his/her spinal posture position is correct. Referring to the drawings, FIG. 1 thereof shows an exemplary first image (preferably a green image) that is viewed by a user whose spinal posture is ergonomically correct, with the neck and head in an upright position. As represented by this first image the user clearly is given clear feedback that the user's spinal posture is correct. In addition, providing this first image in green further reinforces to the user that his/her spinal posture is completely correct.

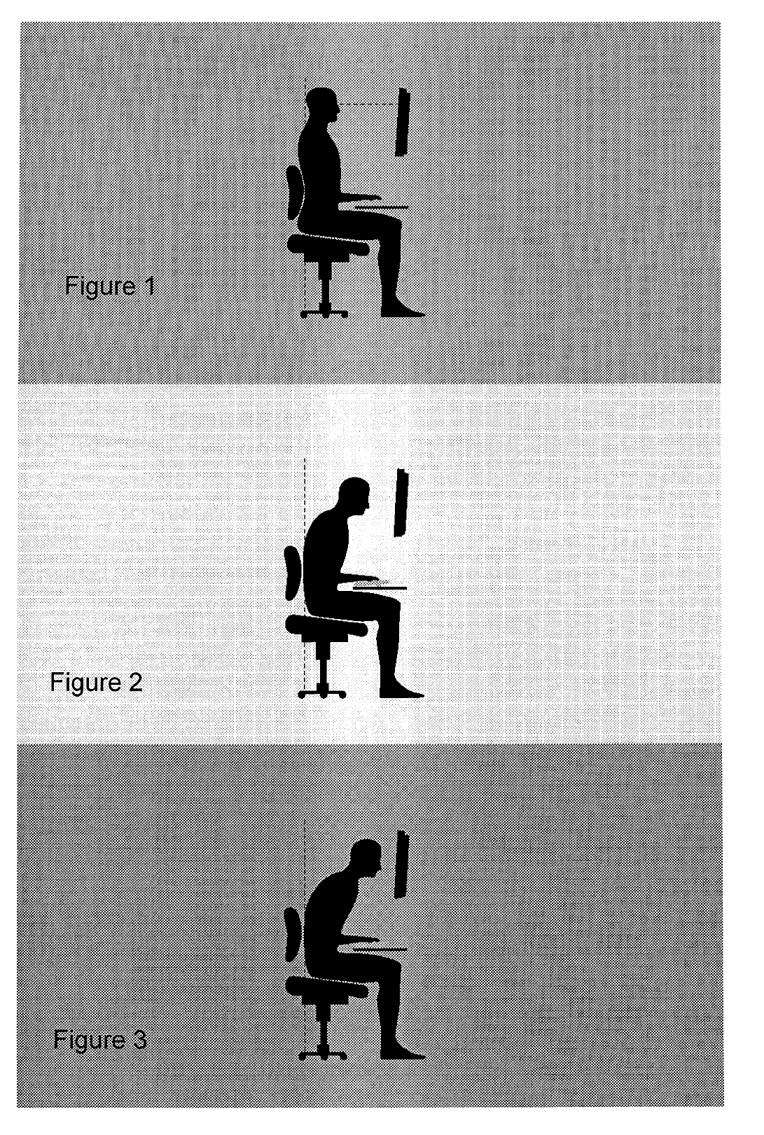

If the user begins to drift away from the proper spinal and posture alignment position, in which the neck and head move forward and downward, the image seen in the optical device transitions to a second, yellow image thereby providing to the user feedback that the user needs to exercise caution as to his seating angle and spinal posture position. In other words, the user should make a small positional correction to his/her spinal posture upon viewing the second, yellow image, and upon making such small positional posture correction, the user should again see the first, green image. FIG. 2 shows an exemplary second image (preferably a yellow image) that is viewed by the user whose spinal posture is ergonomically partially incorrect and as represented by such second image the user clearly is conveyed with feedback that the user's spinal posture is partially incorrect.

If the user's spinal posture drifts or otherwise moves to an ergonomically severely incorrect spinal posture, with the neck and head even lower and more forward, the user sees within the optical device a third, red image thereby indicating to the user that his/her spinal posture is severely incorrect and needs to be modified significantly (or, at least, somewhat more than if the user sees the second, yellow image). FIG. 3 shows an exemplary third image (preferably a red image) that is viewed by the user whose spinal posture is ergonomically incorrect. This third image when seen by a user clearly conveys to the user that his/her spinal posture is fully incorrect and requires significant modification.

Each of FIGS. 1-3 contains a broken line indicating the preferred spinal alignment.

In the above description of the optical device the viewer sees any one of three different images or colors, for example, depending on the user's spinal posture or angular viewing position of the optical device. The optical device however may employ only two images, or colors or more than three images or colors. In addition, the size of each of the lenses as well as the total number of lenses of the lenticular lens of the optical device is chosen to accommodate a standard distance between a computer user and a computer monitor so that the images seen by the user appropriately change with the changes in the user's spinal posture as herein described. Different versions of the optical device each having different sized lenses, may be provided whereby a user can select a version based on the user's preferred distance between himself and the monitor.

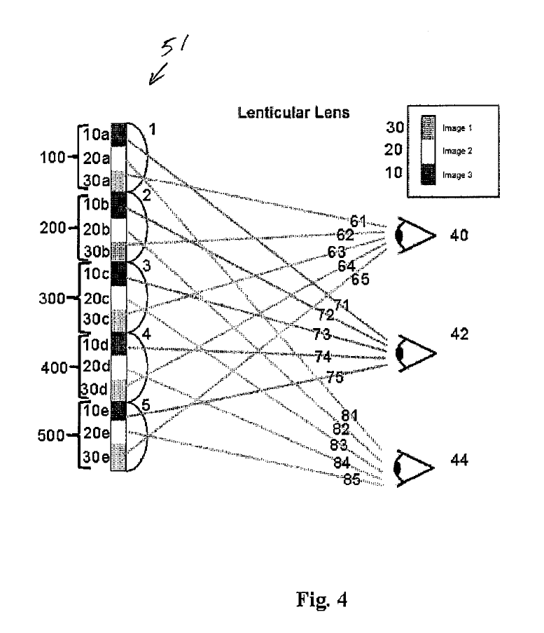

FIG. 4 is a schematic illustration that depicts the operation of the optical device of. The optical device includes three different images, the first, second and third images mentioned above (i.e., as shown in FIGS. 1, 2 and 3). The first image (10) is composed of five (but not limited to five) different image segments 10a, 10b, 10c, 10d and 10e.

Similarly, the second image (20) is composed of five (but not limited to five) different image segments 20a, 20b, 20c, 20d and 20e. Finally, the third image (30) is composed of five (but not limited to five) different image segments 30a, 30b, 30c, 30d and 30e.

When the user is sitting with an ergonomically correct spinal posture, the position of the user's eyes is represented by eye 40 in FIG. 4 and, in such position relative to the position of the optical device, the user views only the first image (i.e., FIG. 1). In such case, lens elements 1-5 focus the user's eye 40 only on image segments 10a-10e that collectively produce the first image. When the user is sitting in a partially incorrect manner, the position of the user's eyes is represented by eye 42 in FIG. 4 and, in such case, the user views only the second image (i.e., FIG. 2). In such case, lens elements 1-5 focus the user's eye 42 only on image segments 20a-20e that collectively produce the second image. When the user is sitting with an ergonomically fully incorrect spinal posture, the position of the user's eyes is represented by eye 44 in FIG. 4 and, in such, the user views only the third image (i.e., FIG. 3). In such case, lens elements 1-5 focus the user's eye 44 only on image segments 30a-30e that collectively produce the third image.

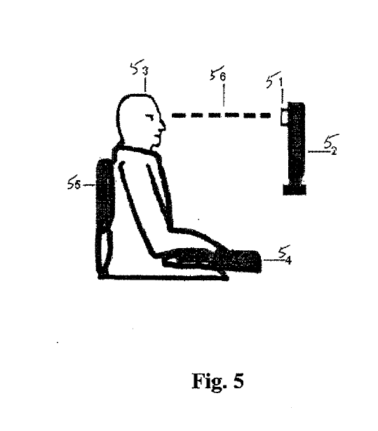

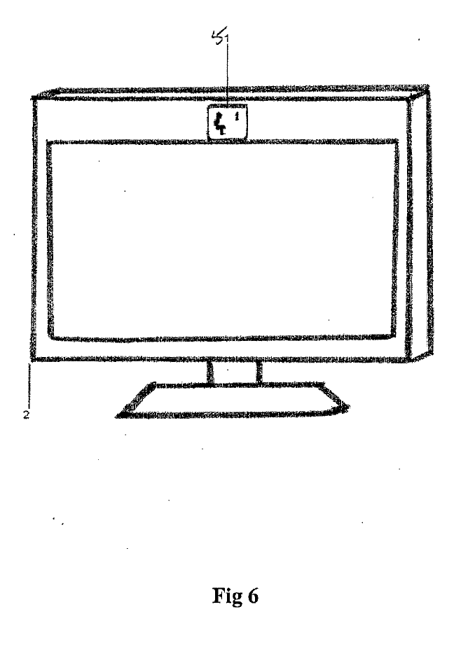

The optical device is placed at a specific position and at an angle on a monitor as determined by the user to give the proper indications according to the position of the head and neck. The optical device may be placed for example at the top center of the monitor's frame. FIG. 5 schematically illustrates a user 53 viewing a monitor 52 with proper placement of the optical device 51. It shows the proper height of the monitor and proper spinal postural relationship of the user. FIG. 6 shows the placement of the optical device 51 on the monitor 52.

As specifically shown in FIG. 5, in the usual case, the optical device 51 is placed at the top of the monitor 52, and is placed at the height of the viewer's eye level, with a direct and straight horizontal line of sight 56 (parallel to the floor line), to the optical device placed upon the monitor. The user 53 is shown seated in a chair 55 in front of a desk 54. Although this is the usual case, different sight-lines or different placement of the optical device may be more appropriate or comfortable for individual users, depending on the particular position or angle of the monitor or chair, for example, or the natural posture of the user.

The height of the monitor and placement of the optical device, may be varied due to the amount of environmental light provided in different surroundings, for example, or the normal position of the monitor, particularly a computer monitor, relative to the vertical position of the viewer's straight line of sight, may not provide the optimum clarity, and thus the optical device preferably can be disposed or mounted on a monitor at a selectable angle. For example, the optical device 51 disposed on the top of a monitor 52 as shown in FIG. 6 may be tilted slightly downward for a monitor disposed higher than that shown in FIG. 5. On the other hand, the optical device may be tilted slightly upward if disposed on a monitor that is positioned below that shown in FIG. 5.

Preferably, the optical device includes an adjustable applicator for enabling the user or other person to mount the optical device on a monitor at a desired viewing angle. The applicator may be an integral part of the optical device or be a separate component altogether. For example, the applicator may be foam tape with a characteristic that allows for the attached optical device to be angularly adjustable. Other suitable adhesives or even mechanical components may be employed as the applicator, such as a spring clip or a hook-and-loop fastener.

During installation of the optical device or during adjustment of an already installed optical device, the angular position of the device may need to be calibrated in order for the proper image to be viewed during the various relative ergonomic postures of the user. Calibration may be carried out by simply tilting the optical device (after being adhered or otherwise connected to a monitor) to a position whereby the user sees the first (green) image when the user is sitting in the optimum ergonomically correct position. Re-calibration may be required if the user's chair 55 is raised or lowered by any appreciable amount or when the user employs a different height new chair or a different size monitor.

Moreover, the user can take the optical device with him/her to another computer, attach the optical device to the top of the monitor, quickly calibrate as described above, and then begin using the other computer while being provided with visual feedback of the user's spinal alignment and posture. As appreciated, installation and calibration of the optical device may take as little as several seconds, thereby making the optical device highly beneficial and convenient, even for individuals who utilize multiple computers.

As described herein, the optical device provides a user seated in front of a monitor with continuous visual feedback regarding the user's spinal posture. The optical device is easily installed to any type of monitor, is easily calibrated and is easily utilized by a user whereby the optical device provides easy-to-understand visual feedback to the user as to the correctness of the user's spinal alignment and posture.

Although particular embodiments have been described, many other variations and modifications and other uses will become apparent to those skilled in the art. Therefore, the present invention is not limited by the specific disclosure herein.

What is claimed is:

1. A method of providing a user with a visual indication of a viewing angle defined between the user's eyes and a target being viewed, comprising the steps of:
providing a device whose appearance changes as a function of the angle at which it is viewed by a user; and
instructing the user to place the device on the viewing target.

2. The method of claim 1, wherein said device shows a first indicator when viewed at a first angle and a second indicator when viewed at a second angle.

3. The method of claim 1, wherein said device includes a lenticular lens.

4. The method of claim 2, wherein said first indicator indicates that the viewer is viewing the target at a most preferred viewing angle, and said second indicator indicates that the viewer is viewing the target at a less preferred viewing angle.

5. The method of claim 4, wherein said first and second indicators include different respective colors.

6. The method of claim 5, wherein said first and second indicators include different respective designs.

7. The method of claim 4, wherein said first and second indicators include different respective designs.

8. The method of claim 4, wherein said device includes a lenticular lens.

9. The method of claim 4, wherein said device shows at least one additional indicator that indicates that the target is being viewed at a least preferred viewing angle.

10. The method of claim 4, wherein said first and second indicators correspond respectively to a higher position and a lower position of the user's eyes.

11. A method of viewing a target with a visual indication of a viewing angle defined between the viewer's eyes and the target being viewed, comprising the steps of:
obtaining a device whose appearance changes as a function of the angle at which it is viewed by the viewer; and
placing the device on the viewing target.

12. A method of providing a computer user seated at a first predetermined position with a continuous visual indication of the user's spinal posture with respect to the computer monitor, comprising the steps of:
providing a device having either a first appearance or a second appearance as a function of the angle at which it is viewed by the user, the first appearance being indicative of a viewing angle with a most preferred spinal posture and the second appearance being indicative of a viewing angle with a less preferred spinal posture; and
instructing the user to place the device on the computer monitor in a second predetermined position so as to show the first or the second appearance to the user in response to the user's spinal posture.

13. A method for a computer user seated at a first predetermined position to view a computer monitor with a continuous visual indication of the user's spinal posture with respect to the computer monitor, comprising the steps of:
obtaining a device having either a first appearance or a second appearance as a function of the angle at which it is viewed by the user, the first appearance being indicative of a viewing angle with a most preferred spinal posture and the second appearance being indicative of a viewing angle with a less preferred spinal posture; and
placing the device on the computer monitor in a second predetermined position so as to show the first or the second appearance to the user in response to the user's spinal posture.

14. The method of claim 13, wherein said device includes a lenticular lens.

15. A device for providing a computer user seated at a first predetermined position with a continuous visual indication of the user's spinal posture with respect to the computer monitor, comprising:
the device showing either a first indicator or a second indicator as a function of the angle at which it is viewed by the user, the first indicator being indicative of a viewing angle with a most preferred spinal posture and the second indicator being indicative of a viewing angle with a less preferred spinal posture; and
the device, when placed on the computer monitor in a second predetermined position, showing the first or the second indicator to the user in response to the user's spinal posture.

16. The device of claim 15, wherein said first and second indicators correspond respectively to a higher position and a lower position of the user's eyes.

17. The device of claim 15, wherein said first and second indicators include different respective colors.

18. The device of claim 17, wherein said first and second indicators include different respective designs.

18. The device of claim 15, wherein said first and second indicators include different respective designs.

20. The device of claim 15, wherein said device includes a lenticular lens.
